(19) United States
(12) Patent Application Publication
(10) Pub. No.: US 2023/0018158 A1
(43) Pub. Date: Jan. 19, 2023

(54) PRE-TEMPLATED INSTANT PARTITIONING OF DNA-ENCODED LIBRARIES

(71) Applicant: FLUENT BIOSCIENCES INC., Watertown, MA (US)

(72) Inventors: Catherine Kugler, Watertown, MA (US); Robert Meltzer, Belmont, MA (US)

(21) Appl. No.: 17/864,655

(22) Filed: Jul. 14, 2022

Related U.S. Application Data
(60) Provisional application No. 63/222,135, filed on Jul. 15, 2021.

(51) Int. Cl. C12N 15/10

(52) U.S. Cl. CPC C12N 15/1065

(57) ABSTRACT

This disclosure provides a powerful screening platform that combines pre-templated instant partitions with DNA-encoded library (DEL) technologies to identify target small molecule interactions and analyze their intracellular effects in single cell resolution using methods that require minimal sample preparation and affordable sequencing costs.

### TECHNICAL FIELD

This disclosure relates to systems and methods for screening and assessing transcriptional effects of DNA-encoded libraries.

### BACKGROUND

DNA-encoded libraries (DELs) offer a powerful tool for screening target-specific chemical compounds. In essence, DELs are small molecules (e.g., drugs) linked with unique DNA tags, which enable facile identification of the small molecules to which they are attached. DELs are especially valuable during initial stages of drug discovery for their ability to identify target-specific small molecules in high throughput. For example, DELs containing billions of small molecules can be rapidly screened in a single experiment to reveal novel drugs that bind with a target of interest.

DELs are useful for identifying target-specific small molecules. Unfortunately, there is no guarantee those molecules will have desirable therapeutic effects. Investigating the transcriptional effects of small molecules on cells is therefore highly desirable. Measurements of gene expression can provide insights on capabilities of small molecules for eliciting desired intracellular effects. Unfortunately, the analysis of even a handful of small molecules on gene expression requires a tremendous amount of sample preparation and sequencing, making such approaches prohibitively expensive.

### SUMMARY

This invention provides a robust platform for screening target-specific small molecules and assessing their intracellular effects in single cell resolution. The invention combines pre-templated instant partitions with DNA-encoded library (DEL) technologies for workflows useful to analyze transcriptional effects of small molecules on single cells with minimal sample preparation and affordable sequencing costs. Accordingly, these methods are well suited for rapidly evaluating drug candidates by simultaneously detecting novel target-binding molecules while screening their transcriptional effects with a single affordable, high throughput workflow. The pre-templated instant partitions enable near instantaneous self-assembly of uniform partitions that isolate single cells for the individual analysis of ligand-target complexes in high throughput in a single tube format. Each partition serves as a “reaction chamber” for preparing gene expression libraries from single cells on a massively parallel scale. Preparing the libraries involves capturing and barcoding transcriptional output (mRNA) of each single cell together with a DEL-member specific sequence (DNA tag), thereby linking transcriptional output of cells with identities of the small molecules with which the cells were treated. Data produced by sequencing the libraries are useful to identify ligand-target interactions with their corresponding intracellular effects all from a single reaction. In addition, the invention provides cost-saving strategies that substantially reduce sequencing burdens for single cell sequencing analysis. The strategies involve workflows that selectively process DELs and/or genes of high interest, thereby focusing sequencing resources on molecules most likely to produce useful results. Accordingly, the invention provides rapid, cost-effective approaches for screening small molecules and assessing their intracellular effects, which is useful for applications ranging from drug discovery to basic research.

The invention provides affordable single cell reaction systems that open new opportunities for drug discovery and expand applications for DEL technologies. Methods of the invention make use of pre-templated instant partitions to enable small molecule screens against targets that cannot be expressed and/or purified in a functional form. As such, methods of the invention provide useful strategies for screening small molecules against challenging target classes that are refractory to conventional DEL investigations due to insolubility, instability, or intrinsic disorder, e.g., ion channels, receptors, transcription factors, protein complexes, and signal-transduction pathways.

In one aspect, this disclosure provides a method useful for screening small molecule interactions and analyzing their intracellular effects on single cells. The small molecules involve drugs or other biologically active compounds with potential to elicit an intracellular effect. For example, the small molecules may be drug candidates against cell surface receptors, e.g., ion channels, G-protein coupled receptors, tyrosine kinase receptors, etc. The small molecules are delivered to the cells in the form of DELs. Accordingly, each one of the small molecules is linked with a unique, amplifiable DNA tag useful for identifying the small molecule to which the DNA tag is attached. Methods of the invention involve binding DELs to a population of live cells. In preferred embodiments, the DELs bind with extracellular receptors present on cell surfaces.

Methods of the invention make use of pre-templated instant partitions to isolate and analyze individual DEL-target interactions in high throughput. The pre-templated instant partitions involve hydrogel template particles that template the formation of a large number (e.g., thousands, millions, or more) of partitions simultaneously, in a single tube, and segregate single cells inside those partitions for single cell analysis. A substantial number of the single cells will be bound with DELs. Each partition provides an isolated compartment for preparing a gene expression library, which provides useful expression data for identifying target DEL interactions and analyzing their transcriptional consequences.

Specifically, after binding DELs with cells, methods of the invention involve combining the cells with template particles that are linked with capture oligos. The template particles enable near-instantaneous self-assembly of single cells into uniform partitions, i.e., droplets, using minimal reagents and without expensive microfluidic devices. The cells and template particles are combined in a tube containing a first fluid, e.g., an aqueous fluid, second fluid (e.g., an oil) that is immiscible with the first fluid is added to the tube. The cells are isolated into individual partitions by shearing the immiscible fluids to create a plurality of partitions, near simultaneously, inside the tube, wherein a substantial number of the partitions contain a single one of the cells and a single one of the template particles.

The single cells are lysed inside the partitions. Gene transcripts (i.e., mRNA) are released from the single cells (i.e., gene transcripts) and are captured and barcoded along with DNA tags inside the partitions with capture oligos attached to the template particles. The barcodes assign unique sequence information to the mRNA and DNA tags of single cells, thereby forming traceable associations between the mRNA and DNA tags to specific cells. Methods of the invention use barcoded oligos to link transcriptional output from single cells with DNA tags that identify small molecules responsible for the transcriptional output.

Because the information of mRNA and DNA tags are linked, methods of the invention provide a useful tool for understanding phenotypic and gene expression changes that result from the binding of DELs. In preferred embodiments, those phenotypic and gene expression changes are uncovered by analyzing sequencing data produced by sequencing gene expression libraries formed from the barcoded mRNA and DNA tags.

Methods of the invention provide useful methods that reduce sequencing burdens of single cell sequencing. According to some embodiments, methods involve pre-screening DELs in one or more binding assays with a target of interest (e.g., cells or proteins). Because DELs must bind with their target to elicit transcriptional responses, pre-screening DELs for target binding affinities is useful to focus sequencing resources on DELs most likely to elicit desired cellular responses while reducing costs of processing single cell libraries of DELs unlikely to elicit any response.

Accordingly, methods of the invention provide workflows useful for identifying DELs with high binding affinities to targets of interest. The binding affinities can be assessed by binding assays that involve combining candidate DELs with targets of interest. The targets can be any target of interest, such as, cells, proteins, viral epitopes, peptidoglycans, etc. The DELs are combined and incubated with targets under conditions that promote binding. After binding, methods involve enriching for candidate DELs that bound with the targets, e.g., by washing away any DELs that failed to bind with the target of interest. Methods further involve identifying a subset of the enriched candidate DELs, wherein the subset includes a portion of the enriched candidate DELs that have a higher binding affinity for targets of interest than a second portion of the enriched candidate DELs.

Identifying DELs with high binding affinities may involve sequencing portions of DNA tags of the enriched candidate DELs. The DNA tags include certain sequence information, including, a first PCR primer binding site, a DEL specific barcode, a unique molecular identifier, and a second PCR primer binding site. The DNA tags can be amplified with sequencing adapters and sequenced using any method known in the art, for example, by next generation sequencing. Sequencing the DNA tags generates sequencing reads. The sequencing reads can be de-duplicated using the unique molecular identifiers and quantified, i.e., counted. A higher number of unique reads corresponds with a higher target affinity of the small molecule to which the DNA tag was attached.

Methods of the invention may include treating cells with DELs pre-selected for their target-binding affinity. Methods may involve screening thousands to millions of small molecules with one or more binding assays and from those assays qualifying the top percentage of DELs based on their target affinities for use in single cell analysis.

In addition, methods of the invention provide strategies for selective sequencing of gene transcripts predicted to be most informative of a cellular response. For example, in some instances, specific gene expression pathways are associated with the regulation of a DEL target. Gene expression analysis of genes involved in pathways of a DEL target can provide useful insight into the transcriptional response provoked by a small molecule. Genes involved in any targeted gene expression pathway can be pre-identified by either analyzing existing data from genome databases or performing a preliminary gene expression analysis, for example, with a microarray. After identifying genes of interest, methods of the invention may involve designing gene specific primers to amplify captured gene transcripts before sequencing.

The capture oligos are preferably linked to an exterior surface of the template particles. Each capture oligo can be linked by a covalent acrylic linkage at a 5′ end and include a free 3′ ends for the capture and barcoding of mRNA and DNA tags from single cells. The capture oligos will generally include one or more of barcodes, primer binding sequences, and molecular binders for capturing mRNA or DNA tags released from single cells. At least a portion of the capture oligos may include molecular binders comprising poly-T capture sequences. Alternatively, the oligos may include molecular binders having sequences complementary to a portion of specific gene transcripts. For example, the template particles may have a plurality of oligos with molecular binders complementary to a panel of specific genes, such as, genes involved in a cell-signaling pathway targeted by the DELs.

### BRIEF DESCRIPTION OF DRAWINGS

FIG. 1 shows a single cell method for screening transcriptional effects of DELs.

FIG. 2 shows a method for single cell analysis of DELs.

FIG. 3 illustrates target capture of mRNA and a DNA tag with a template particle.

FIG. 4 shows an exemplary DEL molecule.

### DETAILED DESCRIPTION

This disclosure provides pre-templated instant partitions for screening small molecule interactions and assessing their intracellular effects on single cells. The pre-templated instant partitions are useful to rapidly prepare single cell libraries from cells treated with DELs in a rapid, high throughput format. The pre-templated instant partitions enable the simultaneous assembly of a nearly limitless number of single cell transcriptome libraries from cells treated with DELs without expensive microfluidic devices. Rather, the pre-templated instant partitions make use of pre-made hydrogel template particles that serve as templates that cause water-in-oil emulsion droplets to form when mixed in water with oil and vortexed or sheared. The template particles template the formation of a plurality of partitions simultaneously, in a single tube, while segregating single cells treated with DELs inside those partitions for gene profiling. For example, an aqueous mixture can be prepared in a reaction tube that includes template particles and DEL treated cells in aqueous media (e.g., water, saline, buffer, nutrient broth, etc.). An oil is added to the tube, and the tube is agitated (e.g., on a vortexer aka vortex mixer). The particles act as templates in the formation of monodisperse droplets that each contain one particle in an aqueous droplet, surrounded by the oil.

The droplets all form at the moment of vortexing—essentially instantly as compared to the formation of droplets by flowing two fluids through a junction on a microfluidic chip. Each droplet thus provides an aqueous partition, surrounded by oil. An important insight of the disclosure is that the particles can be provided with reagents that promote useful biological reactions in the partitions and even that reverse transcription can be initiated during the mixing process that causes the formation of the partitions around the template droplets.

Accordingly, the partitions provide individual reaction compartments with single cells and reagents enclosed therein. The individual reaction compartments are useful for generating single cell gene expression libraries in a parallel format. The libraries are prepared with barcoded oligos introduced into the partitions by template particles. The barcoded oligos include template specific barcode sequences and therefore, are useful to link expressional output (mRNA) of the single cells with identities of corresponding small molecules, via DNA-tags, upon hybridization to the barcoded oligos.

Methods of the disclosure are also useful in making a cDNA library. A cDNA library may be a useful way to capture and preserve gene expression information from RNAs present in single cells with bound DELs. For example, a sample that includes one or more intact cells bound with DELs may be mixed with template particles to form a partition (e.g., droplet) that includes the DEL-bound cell. The cell can be lysed and mRNAs and DNA tags of corresponding DELs can be barcoded. The mRNA can be reverse transcribed into cDNAs either inside or outside partitions. The DNA tags can be amplified by a PCR reaction. The PCR reaction can make use of at least one primer specific to a sequence of a capture oligo of a template particle. The PCR reaction can generate an amplicon of the DNA tag. The amplicon will include a barcode sequence from the capture oligo and an identification sequence of the DNA tag specific to a small molecule.

Analysis of sequencing data is useful to provide valuable insight on transcriptional effects of small molecules at the level of single cells, which is useful for discovery of new drugs and fundamental researcher applications, such as, studies of gene expression pathways regulating disease and development. Accordingly, the invention provides cost-effective analytical tools useful for assessing transcriptional effects of small molecules rapidly and at a single cell level, thus providing methods for faster and cheaper analysis of small molecules. In addition, the invention provides useful workflows that reduce burdens of single cell RNA sequencing.

Small molecules (e.g., drugs or other biologically active compounds) are introduced to the cells in the form of DNA-encoded libraries (DELs). DELs are products of encoded combinatorial synthesis and represent millions of distinct small molecules attached to a DNA sequence (i.e., DNA tag) that encodes unique information about the identity and the structure of each library member. DELs are broadly adopted by major pharmaceutical companies and used in numerous drug discovery programs. The application of the DEL technology is advantageous at the initial period of drug discovery because of reduced cost, time, and storage space for the identification of target compounds.

Conventional applications of DELs involve affinity selection assays. Affinity selection (e.g., binding assays) and sequencing are frequently used to identify DEL members that bind a target of interest. However, applications of DELs in affinity selection assays generally involve purified targets, making many important target classes (ion channels, receptors, transcription factors, protein complexes, and signal-transduction pathways) refractory to investigation due to insolubility, instability, or intrinsic disorder.

Methods of the invention are not limited to applications involving purified target proteins, which make certain classes of targets (e.g., ion channels) unassailable by target binding Instead, methods of the invention use pre-templated instant partitions that enable investigations of small molecule interactions with challenging targets that cannot be expressed and/or purified in an active form. Accordingly, methods of the invention enable investigations of DELs against targets present in cellular environments. The targets can include extracellular targets, such as surface receptors, or intracellular targets, such as proteins involved in DNA replication and rep air.

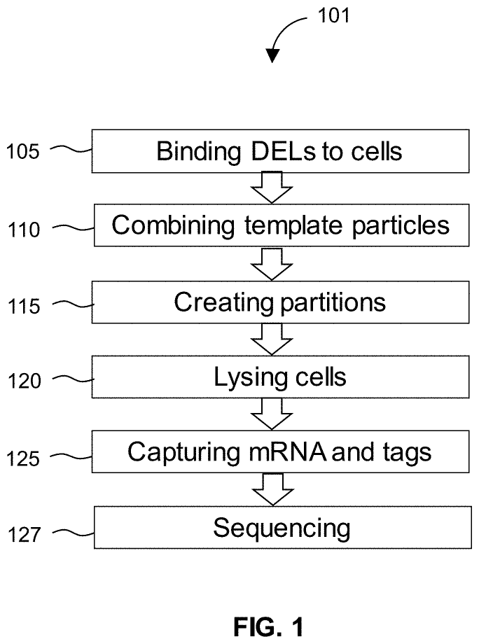

FIG. 1 shows a single cell method 101 for screening transcriptional effects of DELs. The method 101 first involves binding 105 DELs to one or more populations of cells. The cells can be any live cells. The cells can be obtained from cellular tissue taken from a subject. For example, the cells may be tumor cells from a tumor resection. Alternatively, the cells may be tissue culture cells. The cells can be adherent or nonadherent culture cells, such as immortalized cells, e.g., HeLa cells. Without limitation, the cells can be primary cells, stem cells, epithelial cells, endothelial cells, fibroblast cells, or neurons. Preferably, the cells are eukaryotic cells containing a nucleus.

For cell binding 105, the DELs are incubated with the cells under conditions that promote effective target binding. In some embodiments, the target is a cell surface receptor, e.g., an ion channel, G-protein coupled receptor, a tyrosine kinase receptor, or the like. In other embodiments, the target may be an intracellular protein. For those embodiments in which the target is an intracellular protein, a cell-penetrating peptide can be appended to the DELs for delivery of the DELs across the cellular membrane. The DELs may be added directly into a tissue culture flask and incubated with the cells under normal culturing conditions, e.g., at 37 degrees Celsius. The DELs are preferably added at a concentration of approximately 1 DEL per 10 cells, although other concentrations of DELs to cells may be desired, e.g., 1:1, 1:5. 1:15, 1:20, 1:50, 1:100, 1:200, 1:1000. After adding the DELs to the tissue culture flasks, the DELs are incubated for a period of time sufficient for the cells to elicit a transcriptional response to the DELs, which can be between 2-12 hours, and preferably at least 6 hours.

After binding DELs to target cells, the cells are washed to remove any unbound DELs. Washing the cells helps eliminate non-target associations of single cell gene expression and small molecules. The cells can be washed using any cell wash solution commonly used in the art, e.g., a balanced salt solution such as phosphate-buffered saline (PBS). After washing the cells to remove unbound DELS, the cells are removed from the cell culture flask, e.g., using trypsin, and are combined 110 in a tube with template particles inside an aqueous fluid, such as, media or a balanced saline solution. The template particles, as discussed in detail below, involve micron-sized hydrogel particles linked with barcoded oligos useful for capturing and indexing gene transcripts and DNA-tags released by single cells.

In some embodiments, the cells are incubated with the template particles (e.g., for approximately 5-10 min at room temperature) to facilitate surface interactions between the template particles and the cells thereby improving capture of single cells into separate partitions upon shearing or vortexing the mixture. Afterwards, a second fluid that is immiscible with the first fluid is added to the tube. The second fluid is preferably an oil. The second fluid may overlay the aqueous first fluid. In some embodiments, one or more surfactants, described below, may be added to the mixture to stabilize partitions generated by shearing the fluids.

The method 101 involves shearing the fluids to create 115 partitions. Preferably the fluids are sheared by vortexing. Vortexing is preferred for its ability to reliably generate partitions of a uniform size distribution. Uniformity of partitions may be helpful to ensure each “reaction chamber” is provided with substantially equal reagents. Vortexing is also easily controlled (e.g., by controlling time and vortex speed) and thus produces data that are more easily reproducible. Vortexing may be performed with a standard bench-top vortexer or a vortexing device as described in co-owned U.S. patent application Ser. No. 17/146,768, which is incorporated by reference.

Alternatively, creating 115 partitions may involve agitating the tube containing the fluids using any other method of controlled or uncontrolled agitation, such as shaking, pipetting, pumping, tapping, sonication and the like. After agitating (e.g., vortexing), a plurality (e.g., thousands, tens of thousands, hundreds of thousands, one million, two million, ten million, or more) of aqueous partitions is formed essentially simultaneously. Vortexing causes the fluids to partition into a plurality of monodisperse droplets. A substantial portion of droplets will contain a single template particle and a single target cell. Droplets containing more than one or none of a template particle or target cell can be removed, destroyed, or otherwise ignored. Not every single cell will be treated with a DEL and need not be. Sequencing analysis of mRNA and DNA-tags of single cells will reveal the cells treated with DELs based on common, partition-specific barcodes introduced by template particles.

The next step of the method 101 involves lysing 120 the single cells inside the partitions. Cell lysis may be induced by a stimulus, such as, for example, lytic reagents, detergents, or enzymes. Reagents to induce cell lysis may be provided by the template particles via internal compartments. In some embodiments, lysing involves heating the droplets to a temperature sufficient to release lytic reagents contained inside the template particles into the monodisperse droplets.

Upon lysing 120 the cells inside the partitions, mRNA and DNA-tags are released from the cells and into the partitions for capture with the capture oligos provided by template particles. The capture oligos include unique barcodes specific to each template particle. Accordingly, upon capture, i.e., hybridization, of the mRNA and DNA-tags with respective complementary portions of oligos, mRNA and DNA-tags of single cells are effectively linked by a common barcode sequence. Since each partition includes only one single cell and one template particle, the unique barcode sequences of any one template particle is useful for associating mRNA and DNA-tags with single cells from which they are derived.

The oligos are preferably attached to the template particles at a 5′ end with capture sequences (e.g., poly T sequences) at a free 3′ end for target capture of gene transcripts, i.e., mRNA. At least a portion of the oligos include capture sequences that are complementary with at least a portion of the DNA-tags for hybridization capture. The oligos may further include PCR primer binding sites, e.g., a forward and reverse primer binding site for subsequent amplification of the mRNAs and DNA-tags in preparation of sequencing analysis. In addition, the oligos preferably include at least one unique molecular identifier, which enables de-duplication of sequence reads.

After capturing 125, the method 101 may include reverse transcribing captured mRNA into cDNA. Reverse transcription preferably occurs outside of the partitions. As such, the partitions can be broken to release template particles comprising captured mRNA with DNA-tags for reverse transcription. To break the partitions, samples may be treated with a breaking buffer. Once broken, the template particles may be washed with a wash buffer (e.g., ethanol) and bound mRNA and DNA tags may be treated with reagents for reverse transcription to copy mRNA into cDNA with sequence information (e.g., barcodes) provided by template particles. Accordingly, reverse transcription can be carried out to generate a library comprising cDNA with barcode sequences that allows each sequence reads of a library to be traced back to the single cell from which the mRNA and DNA-tags were derived. Once a library is generated comprising barcoded cDNA, the cDNA can be amplified, by for example, PCR, to generate amplicons for sequencing 127.

The DNA-tags preferably include DNA oligonucleotides with functional sequences, such as, one or more PCR primer binding sites, one or more barcodes, and one or more unique molecular identifiers. Following capture of DNA-tags with capture oligos of the template particles, the DNA-tags are amplified by PCR using a first primer complementary to a PCR primer binding site of the capture oligo and a second primer complementary to a PCR primer binding site of the DNA-tag. As discussed below, the PCR primer binding site of the capture oligo is positioned downstream of a template particle specific barcode, which corresponds with the barcodes on capture oligos used for mRNA capture. DNA amplification of captured DNA tags by PCR generates amplicons that incorporate those template-particle specific barcode sequences, thereby associating mRNA and DNA-tags released from a single cell together, by corresponding sequence information useful to trace mRNA and DNA-tags to single cells from which they were released.

The cDNA and DNA amplicons of single cells may subsequently be amplified with sequencing specific primers. For example, such as the sequencing primers used in next generation sequencing (NGS) systems, P5 and P7 sequences. The inclusion of P5 and P7 sequences make the cDNAs and DNA amplicons amenable to sequencing by NGS sequences such as performed on an NGS instrument sold under the trademark ILLUMINA, and as described in Bowman, 2013, Multiplexed Illumina sequencing libraries from picogram quantities of DNA, BMC Genomics 14:46, incorporated by reference.

Sequencing 127 nucleic acid molecules may be performed by methods known in the art. For example, see, generally, Quail, et al., 2012, A tale of three next generation sequencing platforms: comparison of Ion Torrent, Pacific Biosciences and Illumina MiSeq sequencers, BMC Genomics 13:341. Nucleic acid molecule sequencing techniques include classic dideoxy sequencing reactions (Sanger method) using labeled terminators or primers and gel separation in slab or capillary, or preferably, next generation sequencing methods. For example, sequencing may be performed according to technologies described in U.S. Pub. 2011/0009278, U.S. Pub. 2007/0114362, U.S. Pub. 2006/0024681, U.S. Pub. 2006/0292611, U.S. Pat. Nos. 7,960,120, 7,835,871, 7,232,656, 7,598,035, 6,306,597, 6,210,891, 6,828,100, 6,833,246, and 6,911,345, each incorporated by reference. After sequencing, the sequencing data may be subsequently processed for analysis.

One pipeline for processing sequencing data includes generating FASTQ-format files that contain reads sequenced from a next generation sequencing platform, aligning these reads to an annotated reference genome, and quantifying expression of genes. These steps are routinely performed using known computer algorithms, which a person skilled in the art will recognize can be used for executing steps of the present invention. For example, see Kukurba, Cold Spring Harb Protoc, 2015 (11):951-969, incorporated by reference.

Methods of the invention are useful for generating gene expression data from single cells from cells treated with small molecules of DELs. The expression data are useful for making gene expression profiles. In some embodiments, gene expression profiles may be made using analysis tools openly available, such as, TopHat2 (Johns Hopkins University for Computational Biology), Cufflinks (University of Washington, Cole Trapnell Lab), and DESeq2 (See Love M I, Huber W and Anders S, 2014, Moderated estimation of fold change and dispersion for RNA-seq data with DESeq2, Genome Biology, 15, pp. 550, incorporated herein by reference) may be used to align RNA sequences and to determine expression levels and identify differential expression corresponding with DELs. Expression levels may be normalized to expression levels of a housekeeping gene or other control measured in the sample. For example, the normalized expression levels may be compared to a threshold expression level from a single cell not bound or treated with a DEL. A single cell not bound with a DEL can be identified from single cell sequencing data by identifying sequence reads associated with a template-specific barcode for which no DNA-tag barcode sequences exist.

At any moment each cell makes mRNA from only a fraction of the genes it carries. If a gene is used to produce mRNA, it is considered “on”, otherwise it is considered “off”. Gene expression profiling may include measuring the relative amount of mRNA expressed in two or more conditions. For example, cells may be modified by an RNA guide that is thought to produce an “on” switch in a gene, an RNA guide that is thought to produce an “off” switch in a gene, and an RNA guide that is thought to produce no change in the gene. The gene expression profile provides information as to what the changes made by the guide RNAs in DNA actually result in phenotypically in the cell. Gene expression profiling may also provide information about the editing capacity of RNA guides, for example when multiple RNA guides targeting the same “on” switch are analyzed in parallel to assess varying levels of gene expression level changes.

The gene expression profiles provide valuable insight into transcriptional responses of single cells elicited by DEL binding, which are useful for evaluating whether a small molecule elicits a desired or undesired transcriptional effect. The gene expression profiles of single cells treated with DELs can be compared with gene expression profiles of cells not treated to gain an understanding of a phenotypic effect of specific DELs. Moreover, gene expression profiling can be useful for identifying the mechanisms of action of the DELs, e.g., revealing gene pathways that are differentially regulated.

Single cell transcriptomics with multiple small molecules presents significant challenges because of the required sequencing. For example, screening a 10,000 DEL member library against cells with 10× library coverage, and a DEL to cell ratio of, for example, 1:100 with a modest 50,000 sequencing reads per cell would require 500 billion sequencing reads, or approximately 250 NovaSeq lanes, which are each approximately $5,000 USD. As such, single cell sequencing of DELs is prohibitively expensive using existing prior art methods.

Methods of the invention, however, provide useful analytical workflows that provide substantially reduced sequencing burdens by pre-selecting DELs and/or genes of interest for processing. Through pre-selective processing of only those DELs and/or genes of interest, sequencing burdens can be reduced by over 100-fold. For example, from a 10,000 DEL member library, methods of the invention are useful for identifying the top 100 DELs based on their binding affinities for targets of interest. Higher binding affinities of small molecules for targets generally correspond with greater functionalities. 100 DELs at a 100-cell coverage at with a DEL to cell ratio of 1:100 and 50,000 sequencing reads per cell would require 5 billion sequencing reads, or approximately 2.5 NovaSeq lanes, a substantial reduction.

According to one embodiment, methods of the invention are useful to reduce sequencing expenses by pre-screening candidate DELs in one or more binding assays with a target of interest (e.g., cells or proteins). The candidate DELs with highest binding affinities for the target of interest are then selected for single cell gene expression analysis. Because a DEL must bind with a target to elicit a transcriptional response, pre-selecting those DELs with higher binding affinities for target is likely to capture DELs most likely to elicit target responses while reducing costs associated with processing DELs less likely to elicit the target response.

Accordingly, methods of the invention may involve performing a binding assay to identify a subset of candidate DELs based on binding affinity. The target can be any biological target of interest. For example, the target may be a specific protein. The protein may be bound to a solid substrate (e.g., a column). The target may be a specific cell type, e.g., a cancer cell. The target may be a cell surface protein. The target can be a viral epitope, a peptidoglycan, etc.

Methods involve incubating substantially equal amounts of different DEL member libraries with targets of interest under conditions that promote binding. Afterwards, unbound DELs and/or weakly bound DELs are washed away using one or more wash steps to separate unbound DELs and/or weakly bound DELs from target bound DELs. Any known method in the art can be used to separate the DELs by binding their binding affinities. For example, methods may include column washing, cell pelleting, magnetic separations, etc. In some instances, stringency of wash conditions may be adjusted to modify selectivity of the wash. After washing away unbound DELs and/or weakly bound DELs from a substrate, the bound DELs can be released from the target by, for example, alkaline lysis, protease degradation, disulfide reduction, etc. For further discussion on methods for screening DEL interactions see Jonker, 2011, Recent developments in protein-ligand affinity mass spectrometry, Anal Bioanal Chem, 399(8): 2669-2681, which is incorporated by reference.

After screening binding interactions of DELs to a target of interest, those DELs released from the target of interest are further selected by quantification of unique sequencing reads of the bound DELs based on their corresponding DNA-tag sequences. Methods of the invention use sequence read counts to measure and compare target binding affinities of all target bound DELs. A greater number of unique sequence reads correlates with a higher affinity of a DEL member for the target. Sequencing the DNA-tags of released DELs generally involves amplifying unique barcodes from the DNA-tags with sequencing adapters to generate amplicons and subsequently sequencing the amplicons. Sequencing unique DEL barcodes is an efficient sequencing process, requiring only a single 25M read Miseq experiment to produce sufficient sequencing information to identify and compare binding affinities of target bound DELs.

The sequence reads may be de-duplicated based on unique molecular identifiers provided from the DNA-tags. The unique reads for each DEL member can be counted and compared. DEL members with the highest number of sequence reads are identified for further analysis by single cell RNA sequencing analysis.

In another aspect, methods of the invention are useful to reduce sequencing costs of single cell RNA sequencing by selective amplification and sequencing of gene transcripts of interest. For example, in some embodiments, specific gene expression pathways can be targeted for regulation by candidate DELs. In those instances, genes from targeted expression pathways can be amplified and sequenced, sparing massive amounts of sequencing depth needed per cell.

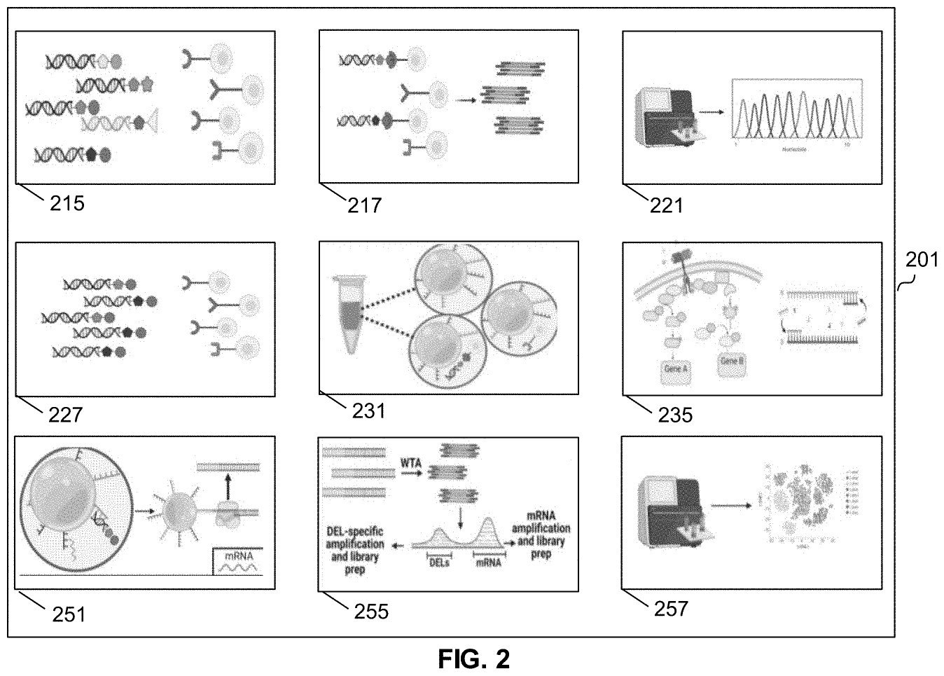

FIG. 2 shows a method 201 for single cell analysis of DELs. The method includes incubating 215 cells with substantially equal amounts of different DELs under conditions that facilitate binding of the DELs with cell surface receptors. Any DELs that do not bind with a target cell surface receptor are washed 217 away by one or more wash steps using a cell wash buffer.

Afterwards, the cells are collected for DNA extraction. DNA can be extracted from the cells by any commercially available DNA extraction kit, such as with a DNA column extraction kit provided by Thermo Fisher. The extracted DNA contains at least a portion of the DNA tags from DELs bound with the cell surface receptors. The DNA tags from the DELs bound with cell surface receptors can then be amplified, e.g., by PCR, with forward and reverse primers that are complementary to primer binding sites of the DNA tags, as discussed below. Amplification of the DNA tags by PCR generates amplicons of DNA tags. The amplicons include unique sequences from the DNA tags which identify the DEL library members from which the tags were derived.

The amplicons are sequenced 221 to generate sequence reads. The sequence reads comprise sequence information identifying those DELs with specificities towards target cell surface proteins. Sequencing 221 can be performed using any sequencer, such as a next generation sequencer provided under the trade name Illumina, and as described in Bowman, 2013, Multiplexed Illumina sequencing libraries from picogram quantities of DNA, BMC Genomics 14:466, incorporated by reference. The sequence reads are analyzed to identify “best binders”. The best binders are DELs having the greatest number of corresponding unique sequence reads.

Next, a fresh batch of cells are incubated 227 with a curated library of DELs comprising the “best binders”. The best binders may, for example, represent the top 5 percent, the top 2 percent, the top 1 percent, or the top 0.5 percent of best binders. After incubation 227, the cells are washed, collected, and prepared for single cell RNA sequencing analysis.

Single cell RNA sequencing analysis requires partitioning 231 the cells into individual partitions, as described in, Hatori, 2018, Particle-templated emulsification for microfluidics-free digital biology, Anal Chem 90:9813-9820, incorporated by reference. Briefly, an aqueous mixture is prepared in a reaction tube that includes template particles and the cells in aqueous fluid (e.g., water, saline, buffer, nutrient broth, etc.). An immiscible fluid (e.g., oil) is added to the tube, and the tube is agitated. The particles act to template the formation of partitions that each contain one template particle in an aqueous droplet, surrounded by the oil.

In some embodiments, methods of the invention involve making 235 gene specific primers to selectively amplify target gene transcripts of interest from the single cells. The genes of interest will generally include reference genes (house-keeping genes) and genes that regulate cellular processes associated with DEL targets. For example, in instances in which the DELs are designed to target a cell surface receptor, the genes of interest may be genes involved in expression pathways of the targeted cell surface receptor. Identifying genes involved in expression pathways of a target receptor can be done by investigating existing gene expression databases, for example, Gene, or the Gene Expression Omnibus database, which are freely available on the web by National Center for Biotechnology Information. Making 235 the primers can be performed by methods known in the art. Alternatively, the primers can be purchased from a third party, for example, the primers for specific genes of interest may be selected and purchased from Biocompare.

The method 201 further includes lysing 251 the cells inside the partitions. Upon lysing 251 the cells, contents of the cells are released into the partitions. Capture oligos linked with the template particles hybridize to mRNA and DNA tags released from the cells. Specifically, poly adenylated portions of mRNA hybridize with portions of capture oligos comprising poly-T sequences. DNA tags hybridize with portions of other capture oligos with complementary sequences. The capture oligos include unique barcodes that link mRNA and DNA tags released from common single cells together. The captured mRNA and DEL DNA tags can be reverse transcribed to create a first-strand cDNA molecule with cell-specific barcode information.

The barcoded first strand cDNA molecules are amplified 255 by whole transcriptome amplification. Whole transcriptome amplification reagents and protocols can be obtained from commercially available kits, such as, the RNA amplification kit sold under the trade name Rapid Amplification of Total RNA, by Sigma. The amplicon products from whole transcriptome amplification reactions are divided into two size specific populations, from which sequencing libraries are. The DELs are preferably amplified separately from the mRNA population to ensure equal representation during sequencing. The DEL and mRNA libraries are then combined 257 in equal proportions and sequenced. The sequence reads are used to generate expression profiles for investigating changes of gene expression in response to small molecules.

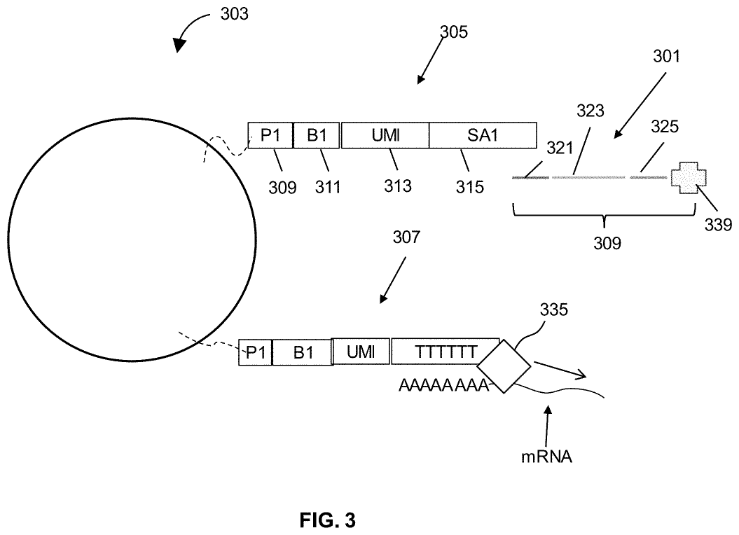

FIG. 3 illustrates target capture of mRNA and a DNA tag 301 with a template particle 303. The capture of mRNA and the DNA tag 301 takes place inside a pre-templated instant partition (not shown), following lysis of a single cell contained therein. The template particle 303 is covalently linked with capture oligos (305, 307). The capture oligos include, from 5′ to 3′, a PCR primer binding site 309, a barcode sequence 311, a unique molecular identifier (UMI) 313, and a molecular binder sequence 315. The template particle 303 includes at least two species of capture oligos 305, 307, with different molecular binders 315. One capture oligo 305 includes a molecular binder with a nucleotide sequence that is complementary with a capture sequence 321 of a DNA tag. A second capture oligo 307 includes a molecular binder with a sequence complementary to mRNA. For example, as illustrated, the molecular binder may comprise a poly-T sequence. Alternatively, the molecular binder may comprise a gene specific sequence. The molecular binders 305, 307 hybridize with the DNA tag 301 and mRNA, respectively, by complementary base pairing.

After hybridization, cDNA synthesis of mRNA attached to template particles is performed with reverse transcriptase 335. Preferably, the partitions are broken before cDNA synthesis. During cDNA synthesis, the reverse transcriptase 335 creates a copy of the mRNA molecule that includes the barcode sequence 311. The barcode sequence 311 comprises a sequence of nucleotides that is unique to its template particle. Accordingly, the barcode sequence 311 allows each library sequence read to be traced back to a common template particle 303.

The DNA tag 301 represents a portion of a DEL 309. A small molecule 339 may be linked at one end of the DNA tag 301. Every DNA tag 301 includes a DEL-member-specific sequence 323. Every DEL member of a library includes an identical small molecule 339. Thus, the sequence 323 contains information useful to identify the small molecule 339 to which it is attached. The DNA tag 301 may further include one or more PCR binding sites 325, and may further include at least one UMI.

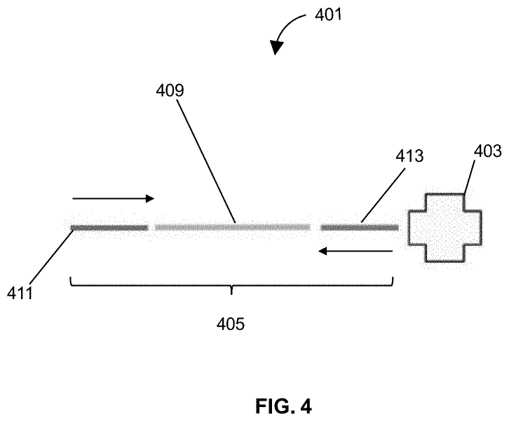

FIG. 4 shows a stylized DEL molecule 401. The DEL 401 includes a small molecule 403 linked with a DNA tag 405. The DNA tag 405 includes at least one DEL barcode sequence 409 that is used to identify the small molecule 403. The DEL barcode sequence 409 is preferably flanked by a capture moiety 411 that is complementary to the PIPs capture moiety (e.g., a Poly-A sequence). The DEL further includes a primer extension site 413. When the DEL is capture by a PIP barcode it can then generate a sequenceable DNA construct. For relative quantitation of DELs, UMIs can be used, but are preferably provided by PIPs. A DEL derived sequencing read can comprise sequence information provided from the PIPs barcode (sequence adapter, PIPs barcode, UMI) and from the DEL (DEL barcode, sequencing adapter).

Methods of the invention are useful to detect ligand-target interactions and assess the cellular consequences of those interactions with an affordable high throughput workflow. Advantageously, methods of the invention are also useful to assess ligand-target interactions in from natural cell environments. Some environments may include intracellular environments. For example, in some instances, cells are treated with DELs that bind intracellular targets, such as, DNA binding proteins, transcription factors, DNA replication machinery, DNA damage proteins, etc., which may represent useful therapeutic targets for controlling cell growth. In those instances, in which intracellular environments are targeted, the DELs may be modified with cell penetrating peptides. Linking cell-penetrating peptides can enable DELs to at least partially cross cell membrane barriers. Cell penetrating peptides typically have an amino acid composition that either contains a high relative abundance of positively charged amino acids such as lysine or arginine or has sequences that contain an alternating pattern of polar, charged amino acids and non-polar, hydrophobic amino acids. The cell penetrating peptides may be hydrophobic peptides, containing only apolar residues with low net charge or hydrophobic amino acid groups that are crucial for cellular uptake.

In other embodiments, DELs are used to target extracellular proteins, such as cell membrane proteins. The targeted membrane proteins may be integral membrane proteins, which are proteins that are a permanent part of a cell membrane and can either penetrate the membrane (transmembrane) or associate with one or the other side of a membrane (integral monotopic). Other proteins targeted may be peripheral membrane proteins, which are transiently associated with the cell membrane. In some preferred embodiments, methods involve targeting membrane receptor proteins. Membrane receptor proteins are important for relaying signals between a cell's internal and external environments.

Methods of the invention are particularly well suited for investigating specific cell signaling pathways. Cell signaling pathways involve cell-cell communication and govern many basic activities of cells. A signal is an entity that codes or conveys information. Biological processes are complex molecular interactions that involve many signals. The ability of cells to perceive and correctly respond to their microenvironment is the basis of development, tissue repair, and immunity, as well as normal tissue homeostasis. Errors in signaling interactions and cellular information processing are implicated in many diseases such as cancer, autoimmunity, and diabetes. By understanding cell signaling, clinicians may treat diseases more effectively and, theoretically, researchers may develop artificial tissues. Methods of the invention allow researchers to perform broad “hypotheses-generating” experiments and produce data that allows researchers to investigate targeted questions about cell signaling pathways and how they are disrupted in disease at the level of the transcriptome.

Methods of the invention are useful to investigate cell signaling pathways. Investigating cell signaling pathways can involve using DELs that target cell surface receptors. Receptors play a key role in cell signaling. Receptors help in recognizing the signal molecule (ligand). Receptor molecules are generally proteins. Receptors may be located at cell surface, or interior of the cell such as cytosol, the organelles and nucleus (especially the transcription factors). Usually the DELs bind membrane-impermeable molecules on surfaces of cells.

Binding the DEL to the target cell surface receptor can cause a conformational change in the receptor, which leads to further transmission of signaling via gene expression pathways. Due to conformational change, the receptor may either show an enzymic activity (called enzymic receptor), or an ion channel opening or closing activity (called a channel receptor). Sometimes the receptors themselves do not contain enzymatic or channel-like domains but they are linked with enzyme or transporter. Some receptors (like the nuclear-cytoplasmic superfamily) have a different mechanism. Once the DELs bind with the receptor, they alter expression of genes within the cell. These alterations are measurable using single cell RNA sequence strategies described herein. Measurements of gene expression are used to identify DELs that produce desired intracellular transcriptional changes. Determining whether a DEL produces a desired intracellular transcriptional change may involve comparing gene expression data with data from a Gene Expression Database, for example, the gene expression profiles of cells treated with DELs may be compared with the Genomics and Drugs integrated Analysis database, which allows researchers to identify whether DELs are active against a disease, such as cancer. For example, as discussed in Caroli, 2018, GDA, a web-based tool for Genomics and Drugs integrated analysis, Nucleic Acids Research, Volume 46, Issue W1, Pages W148-W156, which is incorporated by reference.

The DELs can be designed with small molecules that target cell surface receptors on cells. The receptors may be transmembrane receptors. The DELs are preferably made to target extracellular domains of those receptors. Some receptors targeted by DELs can include G-protein-coupled receptors or single-pass transmembrane proteins. Other receptors may include nicotinic acetylcholine receptors. In some instances, the DELs may target receptors associated with members of the 7™ superfamily.

Methods of the invention are particularly appropriate for identifying potential drug candidates against cell surface receptors, such as, ion channels, G-protein coupled receptors, and tyrosine kinase receptors. Ion channels are pore-forming membrane proteins that allow ions to pass through the channel pore. Identification of drug candidates against ion receptors may be useful for treating channelopathies. G-protein-coupled receptors mediate many physiological responses to hormones, neurotransmitters and environmental stimulants. Mutations in G-protein-coupled receptors can cause acquired and inherited diseases such as retinitis pigmentosa, hypo- and hyperthyroidism, nephrogenic diabetes insipidus, several fertility disorders, and even carcinomas. Methods of the invention can be used to screen target drug candidates against mutated receptors to identify new drugs for treating acquired or inherited diseases.

Receptor tyrosine kinases are the high-affinity cell surface receptors for many polypeptide growth factors, cytokines, and hormones. Receptor tyrosine kinases (RTKs) play an important role in a variety of cellular processes including growth, motility, differentiation, and metabolism. As such, dysregulation of RTK signaling leads to an assortment of human diseases, most notably, cancers. Methods of the invention can combine DEL technologies with single cell analysis workflows using pre-templated instant partitions to screen small molecules that bind with RTKs and elicit desirable transcriptional effects to correct dysregulated RTK signaling pathways and thereby treat underlying disease.

Methods of the invention generally relate to analysis and sequencing of gene transcripts from single cells modified my DELs. Methods may involve analysis of whole genome transcriptomes. Alternatively, to reduce sequencing expenses, methods of the invention may involve selective amplification of mRNA associated with specific genes of interest. In some preferred embodiments, the genes of interest are genes that are involved in gene expression pathways associated with the cell surface receptors being targeted. The genes of interest may be amplified by PCR amplification using gene specific primers. Because each nucleic acid molecule is tagged with a barcode unique to the partition and thus single cell from which it was released, any gene transcript can be traced back to the partition and single cell, thereby allowing for the identification of a genotypic modification created by a specific DEL member.

The template particles may provide oligonucleotides for target capture and barcoding of polyadenylated RNA. Barcodes specific to each template particle may be any group of nucleotides or oligonucleotide sequences that are distinguishable from other barcodes within the group. Accordingly, a partition encapsulating a template particle and a single cell provides to each nucleic acid molecule released from the single cell the same barcode from the group of barcodes. The barcodes provided by template particles are unique to that template particle and distinguishable from the barcodes provided to nucleic acid molecules by every other template particle. Once sequenced, by using the barcode sequence, the nucleic acid molecules can be traced back to the single cell based on the barcode provided by the template particle that the single cell was partitioned with. Barcodes may be of any suitable length sufficient to distinguish the barcode from other barcodes. For example, a barcode may have a length of 4, 5, 6, 7, 8, 9, 10, 11, 12, 13, 14, 15, 16, 17, 18, 19, 20, 21, 22, 23, 24, 25 nucleotides, or more.

The barcodes unique to each template particle may be pre-defined, degenerate, and/or selected at random. Barcodes may be added to nucleic acid molecules by “tagging” the nucleic acid molecules with the barcode. Tagging may be performed using any known method for barcode addition, for example direct ligation of barcodes to one or more of the ends of each nucleic acid molecule. Nucleic acid molecules may, for example, be end repaired in order to allow for direct or blunt-ended ligation of the barcodes. Barcodes may also be added to nucleic acid molecules through first or second strand synthesis, for example using capture probes, as described herein below.

In some methods of the invention, an index or barcode sequence may comprise unique molecule identifiers (UMIs). UMIs are a type of barcode that may be provided to a sample to make each nucleic acid molecule, together with its barcode, unique, or nearly unique. This may be accomplished by adding one or more UMIs to one or more capture probes of the present invention. By selecting an appropriate number of UMIs, every nucleic acid molecule in the sample, together with its UMI, will be unique or nearly unique.

UMIs are advantageous in that they can be used to correct for errors created during amplification, such as amplification bias or incorrect base pairing during amplification. For example, when using UMIs, because every nucleic acid molecule in a sample together with its UMI or UMIs is unique or nearly unique, after amplification and sequencing, molecules with identical sequences may be considered to refer to the same starting nucleic acid molecule, thereby reducing amplification bias. Methods for error correction using UMIs are described in Karlsson et al., 2016, Counting Molecules in cell-free DNA and single cells RNA”, Karolinska Institutet, Stockholm Sweden, incorporated herein by reference.

In some embodiments of the template particles, a variation in diameter or largest dimension of the template particles such that at least 50% or more, e.g., 60% or more, 70% or more, 80% or more, 90% or more, 95% or more, or 99% or more of the template particles vary in diameter or largest dimension by less than a factor of 10, e.g., less than a factor of 5, less than a factor of 4, less than a factor of 3, less than a factor of 2, less than a factor of 1.5, less than a factor of 1.4, less than a factor of 1.3, less than a factor of 1.2, less than a factor of 1.1, less than a factor of 1.05, or less than a factor of 1.01.

Template particles may be porous or nonporous. In any suitable embodiment herein, template particles may include microcompartments (also referred to herein as “internal compartment”), which may contain additional components and/or reagents, e.g., additional components and/or reagents that may be releasable into monodisperse droplets as described herein. Template particles may include a polymer, e.g., a hydrogel. Template particles generally range from about 0.1 to about 1000 μm in diameter or larger dimension. In some embodiments, template particles have a diameter or largest dimension of about 1.0 μm to 1000 μm, inclusive, such as 1.0 μm to 750 μm, 1.0 μm to 500 μm, 1.0 μm to 250 μm, 1.0 μm to 200 μm, 1.0 μm to 150 μm 1.0 μm to 100 μm, 1.0 μm to 10 μm, or 1.0 μm to 5 μm, inclusive. In some embodiments, template particles have a diameter or largest dimension of about 10 μm to about 200 μm, e.g., about 10 μm to about 150 μm, about 10 μm to about 125 μm, or about 10 μm to about 100 μm.

In practicing the methods as described herein, the composition and nature of the template particles may vary. For instance, in certain aspects, the template particles may be microgel particles that are micron-scale spheres of gel matrix. In some embodiments, the microgels are composed of a hydrophilic polymer that is soluble in water, including alginate or agarose. In other embodiments, the microgels are composed of a lipophilic microgel. In other aspects, the template particles may be a hydrogel. In certain embodiments, the hydrogel is selected from naturally derived materials, synthetically derived materials and combinations thereof. Examples of hydrogels include, but are not limited to, collagen, hyaluronan, chitosan, fibrin, gelatin, alginate, agarose, chondroitin sulfate, polyacrylamide, polyethylene glycol (PEG), polyvinyl alcohol (PVA), acrylamide/bisacrylamide copolymer matrix, polyacrylamide/poly(acrylic acid) (PAA), hydroxyethyl methacrylate (HEMA), poly N-isopropylacrylamide (NIPAM), and polyanhydrides, poly(propylene fumarate) (PPF).

In some embodiments, the presently disclosed template particles further comprise materials which provide the template particles with a positive surface charge, or an increased positive surface charge. Such materials may be without limitation poly-lysine or Polyethyleneimine, or combinations thereof. This may increase the chances of association between the template particle and, for example, a cell which generally have a mostly negatively charged membrane.

Other strategies may be used to increase the chances of templet particle-target cell association, which include creation of specific template particle geometry. For example, in some embodiments, the template particles may have a general spherical shape but the shape may contain features such as flat surfaces, craters, grooves, protrusions, and other irregularities in the spherical shape.

In some embodiments, the template particles can be made with DELs. That is, the template particles may be made with DELs incorporated within a hydrogel matrix of the template particle. Template particle carrying DELs can be used to partition cells, as discussed, into droplets. DELs will be released by the template particles allowing single cells to incubated with DELs inside partitions. This is useful for processing DELs and single cells in a 1:1 ratio. Any one of the above described strategies and methods, or combinations thereof may be used in the practice of the presently disclosed template particles and method for targeted library preparation thereof. Methods for generation of template particles, and template particles-based encapsulations, were described in International Patent Publication WO 2019/139650, which is incorporated herein by reference.

### INCORPORATION BY REFERENCE

References and citations to other documents, such as patents, patent applications, patent publications, journals, books, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof

What is claimed is:

1. A method for screening DNA-encoded libraries (DELs) with single cells, the method comprising:
binding cells with DELs comprising small molecules linked to DNA tags;
combining, in a tube containing a first fluid, the cells with template particles comprising capture oligos;
adding a second fluid immiscible with the first fluid to the tube;
shearing the fluids to create a plurality of partitions, near simultaneously, inside the tube, wherein a substantial number of the partitions contain a single one of the cells and a single one of the template particles; and
barcoding, with the capture oligos, mRNA and DNA tags released by the single cells inside the partitions for single cell analysis.

2. The method of claim 1, further comprising pre-screening candidate DELs in a binding assay and, based on the binding assay, selecting the DELs use for cell binding.

3. The method of claim 2, wherein the binding assay comprises:
combining a plurality of candidate DELs with targets;
enriching candidate DELs bound with targets; and
identifying a subset of the enriched candidate DELs for single cell analysis.

4. The method of claim 3, wherein the subset comprises a portion of the enriched candidate DELs having a higher target binding affinity than a second portion of the enriched candidate DELs.

5. The method of claim 3, wherein identifying the subset comprises sequencing DNA tags corresponding with the enriched candidate DELs to generate sequence reads.

6. The method of claim 5, further comprising quantifying the sequences reads of DELs to assess binding affinities, wherein a greater number of unique reads correlates to a higher binding affinity.

7. The method of claim 5, wherein the DNA tags comprise a barcode and a primer binding sequences.

8. The method of claim 1, wherein each one of the DNA tags comprises an oligonucleotide comprising at least one barcode sequence that is unique to the small molecule of the DEL.

9. The method of claim 1, wherein the small molecule comprises a drug candidate against a cell surface receptor.

10. The method of claim 1, wherein the oligos of the template particles comprise one or more of barcodes, primer binding sequences, or molecular binders for capturing mRNA or DNA tags released from cells.

11. The method of claim 10, wherein at least a portion of the oligos comprise molecular binders comprising poly-T capture sequences.

12. The method of claim 1, further comprising amplifying the barcoded mRNA and DNA tags released by the cells to generate amplicons.

13. The method of claim 12, wherein amplifying is performed with gene specific primers to thereby amplify mRNA associated with genes of interest.

14. The method of claim 13, wherein the genes of interests comprise genes involved in gene expression pathways that associated with drug candidates.

15. The method of claim 12, further comprising sequencing the amplicons to produce a plurality of sequence reads.

16. The method of claim 15, wherein the sequence reads comprise sequence information linking the small molecules of DELs with transcriptional output of corresponding single cells.

17. The method of claim 1, further comprising washing the cells after binding.

18. The method of claim 1, wherein the small molecules bind to surface receptors of the cells.

19. The method of claim 1, wherein binding involves integrating at least a portion of the DELs inside the cells.

20. The method of claim 1, wherein the DELs further comprise cell-penetrating ligands.

21. The method of claim 1, wherein the DELs target cell type specific proteins.
